(19) United States
(12) Patent Application Publication
(10) Pub. No.: US 2026/0297491 A1
(43) Pub. Date: Oct. 1, 2026

(54) NEGATIVE DIELECTROPHORETIC CELL FILTER

(71) Applicant: AGILENT TECHNOLOGIES, INC., Santa Clara, CA (US)

(72) Inventors: Curt A. FLORY, Los Altos, CA (US); Dustin CHANG, Mountain View, CA (US); Reid A. BRENNEN, San Francisco, CA (US); Carol T. SCHEMBRI, San Mateo, CA (US)

(21) Appl. No.: 19/479,172

(22) PCT Filed: Apr. 25, 2024
(86) PCT No.: PCT/US2024/026331

§ 371 Date: Oct. 27, 2025

Related U.S. Application Data
(60) Provisional application No. 63/499,112, filed on Apr. 28, 2023.

(51) Int. Cl. C12M 1/26; C12M 1/42; C12N 13/00

(52) U.S. Cl. CPC C12M 33/14; C12M 35/02; C12N 13/00

(57) ABSTRACT

A cell filtration device and method use a negative dielectrophoretic force (nDEP) to repulse cells from pores of an insulating membrane. A cell suspension is introduced to a first fluidic region, and a fluid and other components pass through pores of the insulating membrane to a second fluidic region. Cells are retained in the first fluidic region and can be recovered continuously or at intervals. The cell filtration device and method minimize contact of the insulating membrane by the cells and avoids clogging of pores.

### CROSS-REFERENCE TO RELATED APPLICATIONS

This application is a national stage application, filed under 35 U.S.C. § 371, of International Patent Application No. PCT/US2024/026331, entitled NEGATIVE DIELECTROPHORETIC CELL FILTER, filed on Apr. 25, 2024, which claims priority to US Provisional Application 63/499,112, entitled NEGATIVE DIELECTROPHORETIC CELL FILTER, filed Apr. 28, 2023, the disclosures of which are incorporated herein by reference in their entireties.

### FIELD OF THE INVENTION

The present invention relates generally to devices and methods to filter cells from a cell suspension.

### BACKGROUND OF THE INVENTION

When performing biological assays on cell suspensions, it is often advantageous or required that the first part of the workflow contains some measure of sample preparation. One of the common methodologies that is employed is cell filtration. The ideal cell filter should be capable of collecting and concentrating cells from the cell suspension, washing cells with a wash fluid, and then collecting the washed cells with high efficiency from the filter medium.

Typical cell filtration platforms often suffer from complications that compromise their intended function. Mechanical cell filtration has the limitation that the pores in the filter medium become blocked as cells are trapped, and the flow or throughput of the suspension fluid is reduced. The filtering process can proceed until the number of trapped cells approaches the filter capacity, which is determined by the effective surface area of the filter medium. If this limit is reached, the filter becomes clogged, and the filtration process is self-terminated. Another problem common to mechanical filtration is the difficulty of efficient recovery of the collected cells from the filter medium. The collected cells can often stick to the mechanical filtration medium, or become inextricably lodged in the pores of the structure, and thus remain in/on the filter medium when the filter is backwashed to retrieve the trapped cells. The result is an incomplete recovery of the target cells from the suspension.

There remains a need for a cell filtration platform that has little or no clogging and has a filter capacity that is greatly enhanced over the cross-sectional area of the filter medium. Additionally, it would be advantageous to have easy and efficient cell retrieval from the filtration platform.

### SUMMARY OF THE INVENTION

As an aspect of the present invention, a cell filtration device is provided. The cell filtration device comprises a first fluidic region adapted to receive a cell suspension comprising cells in a medium. The cell filtration device also comprises a porous electrically insulating membrane having first and second membrane surfaces and a plurality of pores, wherein the first membrane surface of the insulating membrane contacts the first fluidic region. The cell filtration device also comprises a second fluidic region adapted to receive a liquid through the insulating membrane, wherein the second membrane surface of the insulating membrane contacts the second fluidic region. The cell filtration device also comprises one or more electrodes positioned such that application of a voltage to the one or more electrodes will generate an inhomogeneous electric field through the pores of the porous electrically insulating membrane.

As another aspect, a method is provided for filtering cells from a cell suspension. The method comprises providing a cell filtration device comprising a first fluidic region adapted to receive a cell suspension comprising cells in a medium; a porous electrically insulating membrane having first and second membrane surfaces and a plurality of pores, wherein the first membrane surface of the insulating membrane contacts the first fluidic region; and a second fluidic region adapted to receive a liquid through the insulating membrane. The second membrane surface of the insulating membrane contacts the second fluidic region. The method also comprises introducing a cell suspension to the first fluidic region of the cell filtration device, wherein the cell suspension comprises cells in a medium. The method also comprises generating an inhomogeneous electric field across the porous electrically insulating membrane such that the cells in the cell suspension move away from the porous membrane. The method also comprises passing at least a portion of the medium of the cell suspension through the porous electrically insulating membrane into the second fluidic region.

These and other features and advantages of the present methods and devices will be apparent from the following detailed description, in conjunction with the appended claims.

### BRIEF DESCRIPTION OF THE DRAWINGS

FIGS. 1A and 1B are conceptual illustrations of a cell filtration device in accordance with an exemplary embodiment of the present disclosure.

FIG. 2 is a conceptual illustration of a dielectric sphere placed in a non-uniform electric field.

FIG. 3 is a conceptual illustration of how a biological cell behaves in a non-uniform electric field.

FIG. 4 is a plot of the Clausius-Mossotti factor calculated as a function of applied frequency.

FIG. 5 is a plot of the Clausius-Mossotti factor calculated as a function of applied frequency where the cell interior is less conductive than the surrounding medium.

FIG. 6 is an elevation view of an embodiment of the present cell filtration device.

FIG. 7 is an exploded view of an embodiment of the present cell filtration device.

FIG. 8 is a photograph of a pore array in embodiment of the present cell filtration device.

FIG. 9 is an illustration of another embodiment of the present cell filtration device, which is suitable for continuous, non-contact filtration of a cell suspension.

FIG. 10 is an illustration of another embodiment of the present cell filtration device, which is suitable for continuous concentration of a cell suspension.

FIGS. 11A and 11B are photographs from a top view and side view, respectively, of a porous membrane comprising a polycarbonate track-etched (PCTE) substrate treated with a hydrophilic coating.

The present teachings are best understood from the following detailed description when read with the accompanying drawing figures. The features are not necessarily drawn to scale. Wherever practical, like reference numerals refer to like features.

### Defined Terminology

It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

As used herein, and in addition to their ordinary meanings, the terms “substantial” or “substantially” mean to within acceptable limits or degree to one having ordinary skill in the art.

As used herein, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same. In the present disclosure, it will be understood that numeric ranges are inclusive of the numbers defining the range.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or.” The terms “includes,” “including,” and “include” are inclusive and have the same scope as “comprises,” “comprising,” and “comprise” respectively.

Unless otherwise indicated, the terms “first”, “second”, “third”, and other ordinal numbers are used herein to distinguish different elements of the present apparatus and methods, and are not intended to supply a numerical limit. For instance, reference to first and second openings should not be interpreted to mean that the apparatus only has two openings. An apparatus having first and second elements can also include a third, a fourth, a fifth, and so on, unless otherwise indicated.

The term “connected” means that two components are fluidically connected, or physically connected, or both. The term “fluidically connected” means that two components are in fluid communication and includes direct connections between the two components as well as indirect connections where one or more other components are in the flow path between the two components. For example, a first component and a second component are fluidically connected if an outlet from the first component is physically connected to an inlet of the second component, or if a conduit connects the first and second components, or if one or more intervening components, such as a valve, a pump, or other structure, is between the two components as fluid flows from the first component to the second component, or vice versa. Components can be physically connected in any suitable way, such as by using ferrules, brazing, and other approaches. In general, physical connections that are fluid-tight and/or that minimize dead-volume are desired for the present devices.

Two or more systems, devices, or components are in “signal communication” when they are capable of communicating with each other via signals that travel over some type of signal path. The signal paths may include physical, electrical, magnetic, electromagnetic, electrochemical, optical, wired, or wireless connections. The signal paths may also include additional systems, devices, or components.

The term “fluidic region” generally refers to a fluidic volume that shares a common purpose, function, or characteristic that distinguishes it from other parts of the extended fluidic volume.

The term “flow path” generally refers to any structure configured to provide for fluid flow. The flow path may be a tube or a channel formed in a substrate. A flow path may be formed by or comprise one or more tubes or channels in fluid communication. A flow path typically has an entrance and an exit, though in some embodiments, a flow path can have multiple entrances and/or exits. The geometry of a flow path may vary widely and includes circular, rectangular, square, D-shaped, trapezoidal or other polygonal cross-sections. A flow path may comprise varying geometries (e.g., rectangular at one section and trapezoidal at another section). In some embodiments, the cross-sectional area of a flow path is substantially constant.

The term “port” encompasses any opening or structure that permits a fluid to pass, including an inlet, an outlet, a conduit, or an aperture or other opening. The term “conduit” generally encompasses any structure such as tubing that defines a flow path for fluid to travel from one point (e.g., an inlet of the conduit) to another point (e.g., an outlet of the conduit), though a conduit can deliver fluid to intermediate points as well. A conduit can be flexible, rigid, or both in some measure or portions. Typically a conduit is relatively long and/or linear and provides a flow path from one component (such as a gas source) to another component.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference in their entireties.

As used in the specification and appended claims, the terms “a,” “an,” and “the” include both singular and plural referents, unless the context clearly dictates otherwise. Thus, for example, “a moiety” includes one moiety and plural moieties.

### DETAILED DESCRIPTION

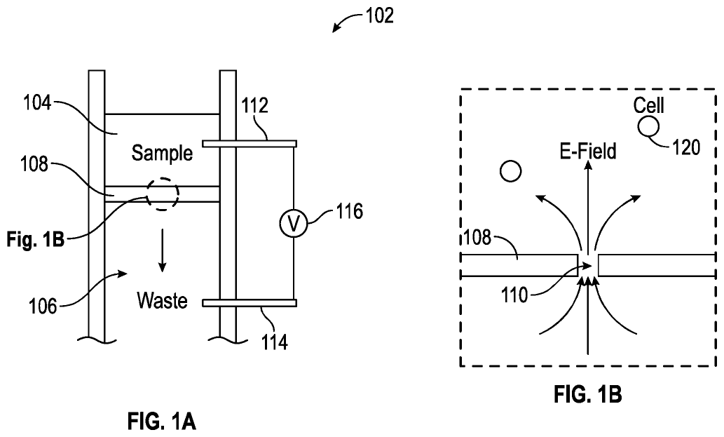

The present invention relates to a cell filtration device that is conceptually and schematically illustrated in FIGS. 1A and 1B. The cell filtration device 102 comprises a first fluidic region 104 and a second fluidic region 106 separated by a porous electrically insulating membrane 108.

A cell suspension is passed through an array of pores 110 in the electrically insulating membrane 108. A voltage is applied across the membrane 108, such as by first and second electrodes 112, 114 which are connected to voltage source 116 through the conducting fluid, and the generated electric current density is highly concentrated as it passes through the pores 110. This implies that a large electric field exists in each of the pores of the array. The first and second electrodes 112, 114 are electrically connected to the porous electrically insulating membrane 108 by the first and second fluidic regions 104, 106, and the porous electrically insulating membrane 108 has an electrical conductivity less than the electrical conductivity of the first and second fluidic regions 104, 106. For appropriate voltage parameters (dependent upon fluid conductivity and permittivity, and cell properties) the cells 120 in the suspension will experience a negative dielectrophoretic (nDEP) force that tends to exclude them from the high-field regions proximal to the pores 110. Fluid from the cell suspension is pushed through the pores of the membrane, the buffer fluid and all extraneous material (non-cells and other debris) will pass through the pores 110 to a waste collection stage such as a vessel, and the cells 120 will be kept from passing through the pores 110 via the nDEP force without contacting the membrane 108. This mechanism of filtration satisfies the desire for a non-clogging filter of enhanced cell capacity, and allows easy and efficient cell retrieval due to the elimination of contact between the cells and the filter medium structure.

The porous electrically insulating membrane 108 can be thin, such as having a mean thickness of 1 μm to 1000 μm. In some embodiments, the porous electrically insulating membrane can have a mean thickness of 10 μm to 100 μm. The porous electrically insulating membrane has pores which can be substantially uniform. In some embodiments, the pores have a mean size of 0.1 μm to 10.0 μm (which can be a diameter or largest dimension of a non-circular pore). The porous electrically insulating membrane can have a characteristic fractional porosity ranging from 0.001 to 0.1.

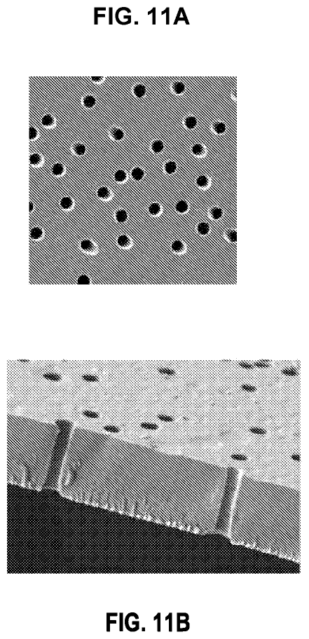

In some embodiments, the porous membrane comprises a polycarbonate track-etched (PCTE) substrate that has been treated with a hydrophilic coating. FIGS. 11A and 11B are photographs from a top view and side view, respectively, of such a substrate. Detailed embodiments and simple calculations showing the parametric conditions under which this platform is operational are disclosed herein. The present device can filter cells from a suspension medium using an imposed nDEP force to preclude one or more of the target cells from entering the pores of the insulating membrane. The device is a non-clogging high-capacity filter that allows efficient recovery of the cells. In order to explain the operational principles and preferred design practices, the present disclosure describes the basic physics behind the dielectrophoretic (DEP) force, as well as the electrical characteristics of biological cells, and the attendant DEP forces that are generated in conducting media. The present disclosure also details nDEP filter embodiments, and provides preliminary experimental results. Finally, the present disclosure contains simple theoretical analysis that can be used to guide and scale the design of operational filters. It is to be understood that the present invention is not limited by theory or to particular embodiments or results.

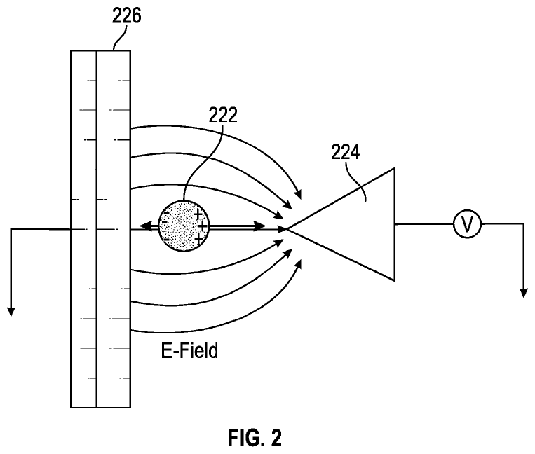

A simple example of a DEP force is illustrated in FIG. 2. In this example, a dielectric sphere 222 is placed in an inhomogeneous electric field created by sharp electrode 224 and plate 226. The surrounding electric field polarizes the dielectric sphere 222, inducing an electric dipole moment. This induced moment interacts with the surrounding field to generate a net force on the sphere 222. If the sphere 222 is more polarizable than the surrounding medium, it is attracted to the high-field regions of the imposed electric field. If the sphere 222 is less polarizable than the surrounding medium, it is attracted to the low-field regions. The force is thus proportional to both the induced electric dipole moment of the sphere 222 and the spatial gradient of the local electric field strength. The equation for the force has the well-known form

$${F}_{d} = 4 {πε}_{m} {ε}_{0} \text{?} ( CM ) {r}^{3} \vert E \vert \vert ∇ E \vert \qquad ( 1 )$$

where εₘ is the relative permittivity of the surrounding medium, ε₀ is the vacuum permittivity, [symbol] (CM) is the usual Clausius-Mossotti factor, r is the sphere radius, and E is the local electric field. For this simple spherical dielectric case, the Clausius-Mossotti factor has the form

$$\text{?} ( CM ) = \frac{( {ε}_{s} - {ε}_{m} )}{( {ε}_{s} - 2 {ε}_{m} )} \qquad ( 2 )$$

where εₛ is the permittivity of the sphere. This shows that the sign of the DEP force changes when the sphere permittivity becomes less than the surrounding medium, and the attraction to high-field regions becomes a repulsion from these same regions.

It is instructive (for present purposes) to note that the relative permittivities can be complex numbers in the usual way, where the real part of the constant corresponds to the normal dielectric constant, and the imaginary part corresponds to the intrinsic conductivity of the specified medium. This generalization is important for a description of the present cell filtration device, as the cells and immersive medium both have a nonzero conductivity associated with them.

### Model of a Cell in Conductive Fluid

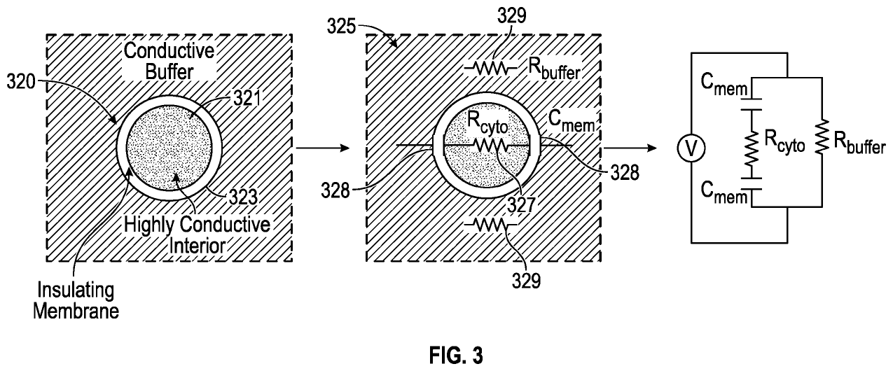

Biological cells do not behave as simple dielectric spheres. However, their physical structure is generically simple enough to allow a relatively simple representative electrical model that qualitatively reflects their behavior in an inhomogeneous electric field, as illustrated in FIG. 3. Specifically, a biological cell 320 has the general form of a (roughly) spherical highly-conductive cytoplasm region 321 that is surrounded by a thin (~5 nm) insulating lipid bi-layer 323. An equivalent circuit model 325 is shown on the right in FIG. 3. For electrical current flowing through the conductive medium surrounding the cell, the physical system can be represented by a parallel circuit with one branch representing the cell with a single resistor 327 (representing the cell interior) isolated from the other branch by connecting capacitors 328 (representing the thin lipid membrane layer), and this branch shunted by the second branch that consists of a single resistor 329 (representing the surrounding conductive fluid).

For DC currents the cell 320 is operationally a non-conductive region due to the insulating outer membrane. As a result, the cell 320 does not generate an effective dipole moment due to the imposed electric fields and is less polarizable than the surrounding medium. Thus, for DC currents, the cell 320 experiences a nDEP force and is repelled from high-field regions.

For an applied RF voltage, the behavior of the cell 320 is more complicated due to the fact that the applied voltage can couple to the (conductive) cell interior 321 capacitively through the thin insulating outer membrane layer 323. Two regimes will be discussed:

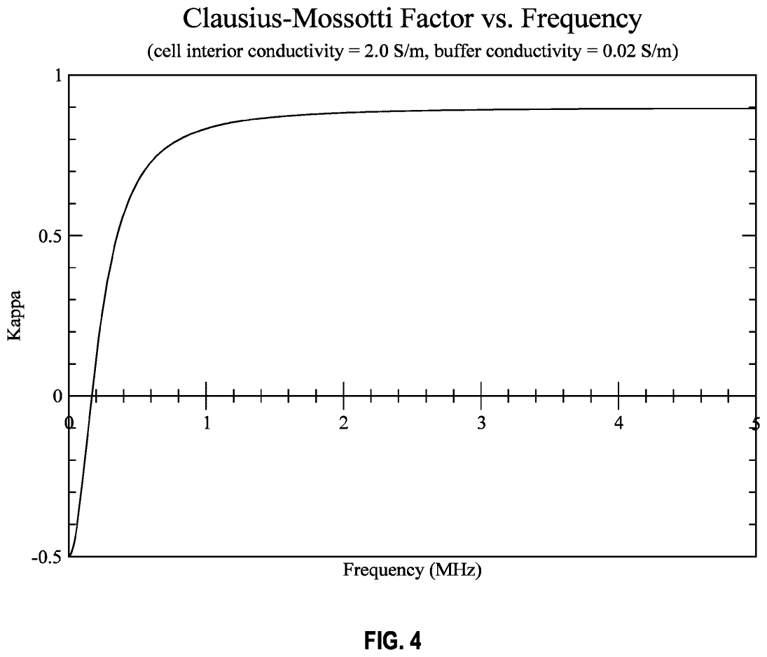

(1) If the cell interior 321 is more conductive than the surrounding medium 325, the cell 320 has the property that, for low frequencies, it has a nDEP response to the applied field, as discussed above (due to inefficient capacitive coupling at “low” frequency), however at a high enough frequency the capacitive coupling to the cell interior 321 becomes adequate to polarize the cell interior 321 to an extent greater than the surrounding medium 325, which leads to a pDEP response. This is reflected in the Clausius-Mossotti factor, which is calculated as a function of applied frequency, and plotted in FIG. 4 (parameter values shown on the figure). FIG. 4 shows that for a frequency below about 200 kHz, the cell behaves like a non-conducting ball with a nDEP effect (C-M factor<0). However, for frequencies above 200 kHz, the Clausius-Mossotti factor is greater than zero, and the cell behaves like a (strongly) conductive entity, and experiences a pDEP force.

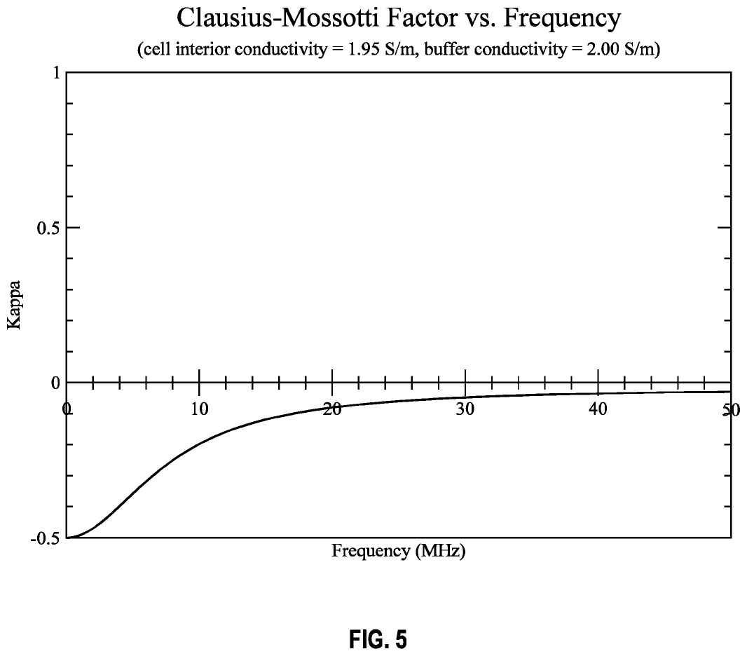

(2) If the cell interior 321 is less conductive than the surrounding medium 325, then for all frequencies, the possible polarization of the cell interior 321 will be less than that of the surrounding medium 325. This leads to a nDEP effect for all frequencies of an applied voltage. For low frequencies (as in the other case) there is virtually no polarization of the cell, and there exists a strong nDEP force (C-M factor<0). As the frequency is raised, there is capacitive coupling to the cell interior and some polarization induced within the cell, but due to the lower conductivity of the cell interior there is merely a reduction of the nDEP force, as it is never as large as that of the surrounding fluid. This behavior is reflected in the Clausius-Mossotti factor, which is calculated as a function of applied frequency, and plotted in FIG. 5 for an example where the cell interior is less conductive than the surrounding medium (parameter values shown on the figure).

For typical cells, the effective conductivity of the cell interior is substantially lower than the conductivity of suspension buffers used to maintain biologically viable cells. Thus, for most applications, the only dielectrophoretic force available to manipulate cells in biologically relevant fluids is the nDEP force. This is the dynamical physical force employed by the cell filtration devices disclosed herein.

nDEP Filter Embodiments

A simple conceptual example of a cell filtration device is illustrated in FIG. 1A. It comprises a first fluidic region or compartment where a cell suspension is introduced, a second fluidic region or compartment for collection of waste fluid and debris or other material, and a thin (electrically insulating) membrane containing an array of pores that is in contact with and separates the two fluidic regions or compartments. Additionally there is a means for applying a voltage of specified amplitude and frequency to the (electrically conductive) fluid suspension, such as one or more electrodes, where the two electrode structures are on opposite sides of the thin membrane. There is also a means for driving the fluid through the array of pores (e.g. syringe pump, pressurized system, etc.). Finally, there is a means to reverse the direction of flow, allowing recovery of the collected cells.

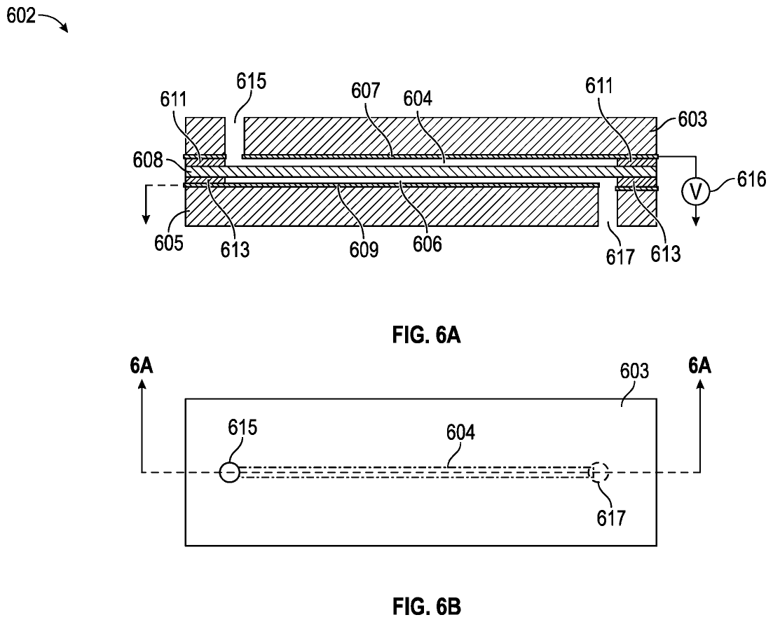

While the conceptual embodiment illustrated in FIG. 1A is a simple platform for describing the elementary functional components of the present cell filtration device, there are practical considerations that guide design parameters. As an example, most of the voltage drop should occur across the thin insulating membrane 108 containing the array of pores 110, rather than across the (finite) resistance presented by the fluid contained in the two fluidic regions 104, 106. The reason for this is that the current flow through the conductive fluid generates heat, which can have deleterious effects on living cells. As a result, it is desirable to limit the length of the path of the current, limiting the total voltage required, and thus the attendant joule heating (detailed below). This necessitates having the electrodes relatively close to the pores 110. While there are many ways that one could implement the (conceptual) nDEP filter, a simple embodiment is a planar structure as illustrated in FIGS. 6A and 6B.

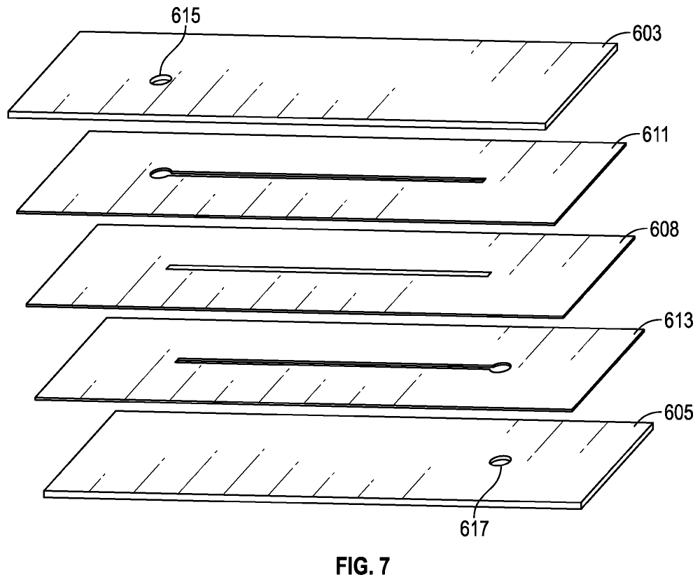

The embodiment of the nDEP filter 602 shown in FIGS. 6A and 6B can be fabricated as a multi-layer sandwich structure. As shown in the figure, the top and bottom layers 603, 605 are rigid substrates that have on their inner surfaces conductive layers 607, 609 that act as an electrode structure, and a thru-hole used as an input and/or output fluidic port 615, 617. The next layer 611, 613 are insulating thin layers that define the dimensions of the first and second fluidic region 604, 606, respectively. Their thicknesses establish the height of the fluidic regions 604, 606, and the pattern cut in these layers 611, 613 define the lateral dimensions of the fluidic regions. The innermost layer 608 comprises the thin electrically insulating membrane that contains an array of pores that allows fluid transfer between the two defined fluidic regions 604, 606. Finally, a voltage source 616 is attached to provide the required voltage drop across the pores. FIG. 7 depicts an exploded representation of the individual layers of an embodiment of the device 602.

The various layers depicted in FIGS. 6A and 6B could take many forms, as will be apparent in view of the present disclosure. For purposes of explanation, and not as a limitation, further details of a particular embodiment, are now provided, but the present device is not limited to this embodiment. The outer layers 603, 605 can be advantageously chosen to be glass slides with a thin layer of metal deposited on the inner surfaces to form the conductive layers 607, 609. For some applications, this thin layer of metal can be indium tin oxide (ITO), to allow observation of the operation of the nDEP filter, although any conductive metal layer would suffice. Examples of other conductive metals, alloys and oxides for the conductive metal layer include aluminum, silver, and gold. Input and/or output holes can be drilled in the substrates, and microfluidic ports can be bonded over the holes to allow attachment of microfluidic capillaries. The insulating layers 611, 613 that are adjacent to the glass slides can advantageously be chosen to be double-sided adhesive tape with a thickness of approximately 100 microns. The tape can be laser-cut to define microfluidic channels (fluidic regions) that are on the order of 1 mm wide, and run a fraction of the length of the glass slide. These pieces of tape can be attached to the respective glass slides, forming 3 sides for each of the respective channels. Finally, the porous electrically insulating membrane 608 can comprise a thin (75 micron) layer of polyimide film, which has an array of pores (each ~3 microns in diameter), where the array covers some or all of the area of the fluidic channels 604, 606 defined by 611, 613 the insulating layers (double-sided sticky tape). When the described layers are aligned and pressed together, the two fluidic channels are completely formed, and they are only connected to one another through the pores on the intervening porous layer.

The present invention is illustrated by, but not limited to, the geometry of the planar device of FIGS. 6A and 6B. Many related structures, using different materials, can also be made and used in view of the present disclosure. All such embodiments would be covered by the conceptual structure illustrated in FIG. 1, which comprises two fluidic regions in fluidic contact through an array of small pores, with an appropriate voltage applied across this array of pores, for the express purpose of excluding the cells of a cell suspension from entering the pores (via a nDEP effect) as the fluid is pumped through the pore array.

The cell filtration devices disclosed herein can be key components of larger systems for creating, handling, maintaining or using cell suspensions. By way of example, the cell filtration devices can be connected to vessels, other filter devices, circulation units, pressurization units (such as pumps), controllers, and others. Accordingly, the present invention also includes systems comprising the cell filtration devices connected to one or more other components.

The present invention can be used to filter or retain cells in a cell suspension, with a greatly reduced potential for clogging over conventional mechanical filtration devices. The cell retention capacity is also enhanced, as is the ability to recover the cells from the filter medium, as the cells never make contact with the filter surface. The range of applications is as broad as that of conventional mechanical filtration systems. The present cell filter device can be used to concentrate a cell suspension in reduced volume, perform cell washing, allow buffer exchange, or extract product from a cell suspension in a bioreactor, to highlight some typical applications.

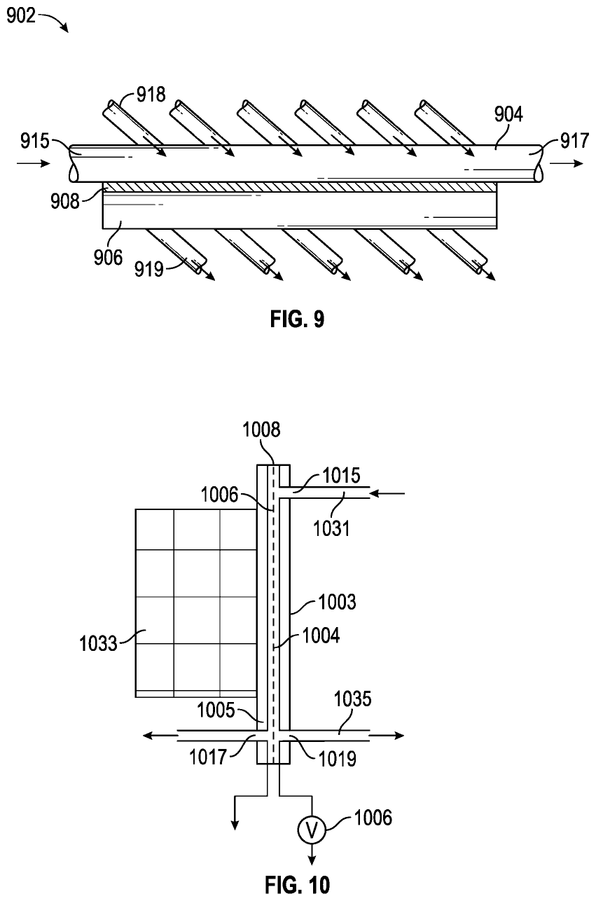

In some embodiments, the present devices and methods can be configured for extracting product from a cell suspension in a bioreactor. For example, the device could be modified to a transverse flow-through geometry, as illustrated in FIG. 9. This transverse flow-through geometry could also be used for cell washing, buffer exchange, and cell concentration.

FIG. 9 shows an embodiment of a transverse flow-through nDEP filter 902. The filter comprises a first fluidic region 904, a second fluidic region 906, a porous electrically insulating membrane 908, and one or more electrodes (not show) for generating an inhomogeneous electric field through the pores of the porous electrically insulating membrane 908. First fluidic region 904 receives a cell suspension from a bioreactor through input fluidic port 915. The cell suspension flows across the porous electrically insulating membrane 908 before passing through output fluidic port 919, which can be fluidically connected to the bioreactor so as to return the filtered cell suspension. nDEP filter 902 also comprises one or more supplemental input fluidic ports 918 for adding a supplemental fluid to the cell suspension such as fresh growth medium. A voltage drop is provided across the pores of the porous electrically insulating membrane 908, generating an inhomogeneous electric field across the porous electrically insulating membrane 908 such that the cells in the cell suspension move away from the porous membrane. At least a portion of the medium of the cell suspension through the porous electrically insulating membrane 908 into the second fluidic region 906, and that portion further comprises one or more bioproducts, contaminants, wastes, or other components. Fluid received in second fluidic region 906 can exit through one or more recovery outlet fluid ports 917 or disposal outlet fluid ports. For example, where the cell suspension comprises cells expressing a desired bioproduct, such as a recombinant protein, the protein can be recovered through recovery outlet fluid ports 917 without disrupting production in the bioreactor. Such embodiments can also provide a benefit of separating large cells from small cells, platelets or lysed cell debris in a cell suspension.

FIG. 10 illustrates another embodiment of the present cell filtration devices and methods. The device is a continuous-flow cell-concentrator 1002 similar to that described in FIGS. 6A and 6B, with the addition of an output fluidic port 1019 at the end of the cell suspension input channel (first fluidic region 1004). This is to allow continuous collection of the concentrated cell suspension as the device 1002 is operated. It can be fabricated using the procedure previously described in FIG. 7. The device 1002 has top and bottom layers 1003, 1005 are rigid substrates that have on their inner surfaces conductive layers that act as an electrode structure. It also have input and output fluidic ports 1015, 1017. The device can include insulating thin layers that define the height and lateral dimensions and/or define a flow path of the first and second fluidic regions. An innermost layer 1008 comprises the thin electrically insulating membrane that contains an array of pores that allows fluid transfer between the first and second fluidic regions 1004, 1006. A voltage source 1016 is attached to provide the required voltage drop across the pores. Fluid received in second fluidic region 1006 can exit through one or more recovery outlet fluid ports 1017 or disposal outlet fluid ports. Concentrated suspension passes through output fluidic port 1019, which can be fluidically connected via a conduit 1035 to a bioreactor so as to return the concentrated filtered cell suspension.

In some embodiments, the device 1002 shown schematically in FIG. 10 can be operated as follows. A cell suspension is driven through an input capillary 1031 using a syringe pump into the first fluidic region 1004 (e.g., a channel). The cells travel down the first fluidic channel 1004 toward the output fluidic port 1019 at the end of the first fluidic channel 1004. As the suspension is transported down the first channel 1004, fluid is continually driven through the porous membrane 1008 to the second fluidic region 1006 (e.g., a waste channel), while the cells are precluded from crossing the membrane by the nDEP force generated by the applied voltage, as described above. The level of cell concentration enhancement is controlled by the amount of fluid that crosses the membrane 1008 to the second channel 1006 during operation. A exemplary device 1002 could have the following dimensions: channel lengths of about 20 mm, channel widths of about 1 mm, channel heights of about 100 micrometers. A Peltier cooler 1033 can be included, as shown in FIG. 10, to mitigate any potential temperature rise of the fluid due to ohmic heating generated by the currents applied to effect the nDEP forces.

In an exemplary device 1002, a porous membrane 1008 as shown in FIGS. 11A and 11B is included. The porous membrane 1008 can have a membrane thickness of about 10 micrometers, a mean pore diameter of about 2 micrometers, and a mean pore density of about 2×10⁶ pores/cm². For such a porous membrane 1008, the total flow resistance of fluid passing through the device is a few orders of magnitude smaller than the flow resistance of several centimeters of capillary with an inner diameter (ID) of 125 micrometers. As a result, by choosing the ratio of capillary lengths connected to the cell suspension output port and the waste output port, one can control the final cell concentration level, as the flow resistances of these capillaries control the rate of fluid flow to the two individual output ports. For example, to obtain a cell concentration enhancement of 10×, one would attach 2 cm of 125 micrometers ID capillary to the waste port, and 18 cm of 125 micrometer ID capillary to the cell suspension output port. For an input cell suspension flow rate of 50 μL/min, an applied voltage of 1-5 Vᵣₘₛ is more than adequate to repel the cells from the membrane pores via the nDEP forces.

### Parameter Design Estimates and Scaling

In designing and optimizing the nDEP cell filter, three dynamical processes are to be analyzed and controlled. These processes are: the nDEP forces that push the cells away from the pores, the fluidic drag forces that drive the cells toward the pores, and the joule heating that occurs in the fluid due to the applied voltage. For the filter to be operational, the nDEP forces must comfortably exceed the fluidic forces on the cells, and the joule heating must not raise the fluid temperature enough to harm the cells. These processes will be estimated in turn for the test device described above. The results can be scaled for other geometries.

(1) nDEP Force Estimate

The E-field inside or near a small pore can be estimated from the voltage drop Vₚₒᵣₑ through the pore, and the diameter d of the pore as

$${E}_{pore} \approx \frac{{V}_{pore}}{d} .$$

Also, the electrical energy density near the pore is given by

$$U = \frac{1}{2} {ε}_{r} {ε}_{0} {E}_{pore}^{2}$$

where εᵣ is the relative permittivity of the fluid and ε₀ is the vacuum permittivity. The electrical force on an approaching cell can be estimated via the principle of virtual work. For the voltage conditions described in the previous sections, the cell acts as a non-polarizable and non-conducting sphere. As it approaches the pore, it replaces the region of polarized fluid (with the attendant electrical energy density given above) by a non-polarized region of reduced energy. The effective force on the cell can then be estimated using the principle of virtual work:

$$\begin{matrix}{F}_{DEP} = - \frac{\partial }{\partial z} ( ( U \times Volume ) ) \\ = - \frac{\partial }{\partial z} ( ( U \times {π ( \frac{d}{2} )}^{2} z ) ) \\ = - \frac{1}{2} {ε}_{r} {ε}_{0} {E}_{pore}^{2} ( π {d}^{2} / 4 )\end{matrix}$$

where z is the distance the cell is above the pore. For a 3 μm pore in water with an applied voltage of V, the force in newtons is given by

$${F}_{DEP} \approx - 2.8 \times {10}^{- 10} {V}^{2} N$$

### (2) Fluidic Force Estimate (on K562 Cell)

For the test device described above, the effective area of the filter is a disk of 1 mm diameter (the area of the pore array), and the flow rate is Q=5 μL/min. Thus, the flow velocity in the extended region near the pore array surface is given by

$${V}_{b} = \frac{Q}{( filter area )} = 1. \times {10}^{- 4} m / s .$$

For laminar flow, the fluid drag on the cell should be given by Stoke's formula

$${F}_{S} = 6 π {r}_{cell} η {V}_{b}$$

Where η=8.9×106⁻⁴ kg/m-s for water and r_{cell}~8 μm for K562 cells, yielding

$${F}_{S} \approx 1.34 \times {10}^{- 11} N .$$

Note that for this test device, the nDEP force pushing the cells away from the pores is substantially larger than the fluidic force driving the cells toward the pores for a modest applied voltage of a few volts. To apply these results to other geometries, one can readily scale the appropriate factors in the given equations.

### (3) Joule Heating Effects

It is straightforward to estimate the power dissipated in the fluid for the test device previously described. The pores that are generated by laser drilling through the polyimide film have a truncated conical structure. The terminal radii (a₁ and a₂) are 1.5 μm and 8.0 μm respectively for the conical hole through the 75 μm polyimide layer. Thus, the electrical resistance of a single pore filled with PBS (1×) (the suspension fluid for the K562 cells, with β_{PBS}=0.67Ω−m) is given by

$${R}_{pore} = \frac{( pore length ) ( {ρ}_{PBS} )}{π {a}_{1} {a}_{2}} = 1.3 \times {10}^{6} Ω .$$

Thus, the pore array resistance (1250 pores) is given by

$${R}_{tot} = \frac{{R}_{pore}}{1250} = 1040 Ω .$$

The power dissipated for an applied 1 V is

$$Power = \frac{{V}^{2}}{{R}_{tot}} = 9.6 \times {10}^{- 4} \frac{J}{s} .$$

The mass flow rate through the device is given by

$$\frac{dM}{dt} = Q \times ( water mass density ) = 5 \frac{μL}{min} \times 1 \frac{g}{{cm}^{3}} = 8.3 \times {10}^{- 8} kg / s .$$

Thus, if there is no heat loss to the surroundings (which is a gross underestimate for such microsystems), the fluid would rise by the following amount due to joule heating

$$Δ T = \frac{Power}{{C}_{V} ( \frac{dM}{dt} )} = 2.76 °C .$$

While this is an overestimate of the temperature rise, it is a reminder to consider joule heating in the design and operation of a nDEP filter with a highly conductive fluid. When safe operational parameters are ascertained for a specific device, these equations can be used to scale the results to other devices.

As another aspect, the present disclosure provides methods for filtering a cell suspension. In some embodiments, the cell suspension is filtered and cells are recovered from the suspension. In other embodiments, the medium or one or more components thereof are recovered from the suspension.

The method can comprise establishing a cell suspension (a cell culture) in a bioreactor or other vessel with cells expressing a desired bioproduct, such as a recombinant protein. The cell culture can be maintained by perfusing fresh cell culture medium into the bioreactor, passing the cell culture through a filter and collecting a permeate which contains one or more of the desired bioproducts.

For a cell suspension containing cells of animal or plant origin in a bioreactor in high density, it is desirable to frequently remove contaminants, such as toxic metabolites. This can be achieved by removing cell-free contaminated medium from the bioreactor and replacing it by fresh medium. For efficiency it is desirable to continuously remove and replace cell medium, but it is undesirable to remove or damage cells while doing so. The present cell filtration device of FIG. 9 is especially advantageous for continuous removal and replacement of the medium.

In some embodiments, the present device and methods are used to recover one or more bioproducts from a cell suspension. For instance, where cells in the cell suspension have been engineered or modified to produce a bioproduct, the bioproduct can be passed into the second fluidic region. Examples of bioproducts which can be recovered include proteins and peptides.

In some embodiment, a pressure differential is created across the porous electrically insulating membrane in order to drive the medium across it. The pressure differential can be created by a pressurization unit fluidically connected to one or both fluidic regions. For example, an increased pressure may be induced in the first fluidic region, and/or a reduced pressure may be induced in the second fluidic region, to create a pressure difference between the first and second fluidic regions. In some embodiments, a circulation unit creates a flow of the cell suspension into and/or out of the first fluidic region. For instance, the circulation unit can create a continuous flow of the cell suspension from an inlet to an outlet of the first fluidic region.

### Example

Test devices have been fabricated using the protocol described above for FIGS. 6A, 6B and 7. One change in the described protocol was to have the laser-drilled pores restricted to a 1 mm diameter region in the polyimide film at the end of the input fluidic channel of the geometry illustrated in FIGS. 6A, 6B and 7. This was to allow direct observation of the suspension cells' interaction with the pore array when a voltage was turned on and off.

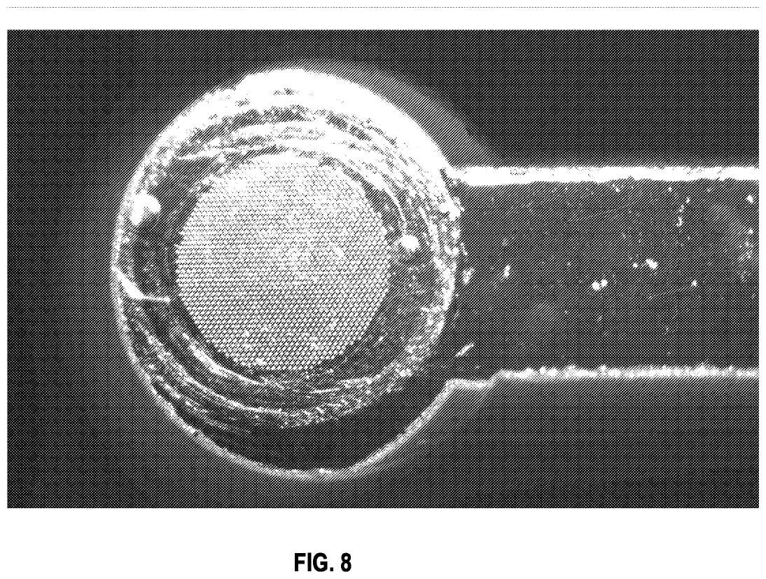

A back-lit image of this pore array is shown in FIG. 8. The pore array has 1250 pores arranged in a hexagonal pattern over a 1 mm diameter area, and is easily seen at the end region of the input fluidic channel. The test device was tested by injecting a suspension of K562 cells (10⁵ cells/cc) at a flow rate of 5 μL/min. With no voltage applied, cells were observed to flow into the array (from the right of FIG. 8), and then disappear through the pores into the output (waste) channel below. When a voltage was applied (10 kHz, 3.5 Vᵣₘₛ), the cells stopped disappearing into pores, and instead remained in the field of view, corroborating the successful use of the test device as a nDEP filter.

### Exemplary Embodiments

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Embodiment 1. A cell filtration device comprising: a first fluidic region adapted to receive a cell suspension comprising cells in a medium; a porous electrically insulating membrane having first and second membrane surfaces and a plurality of pores, wherein the first membrane surface of the insulating membrane contacts the first fluidic region; a second fluidic region adapted to receive a liquid through the insulating membrane, wherein the second membrane surface of the insulating membrane contacts the second fluidic region; and one or more electrodes positioned such that application of a voltage to the one or more electrodes will generate an inhomogeneous electric field through the pores of the porous electrically insulating membrane.

Embodiment 2. The cell filtration device of embodiment 1, wherein application of a voltage to the one or more electrodes will generate an electric field substantially perpendicular to the insulating membrane.

Embodiment 3. The cell filtration device of embodiment 1 or embodiment 2, wherein the one or more electrodes are electrically connected to the porous electrically insulating membrane by the first and second fluidic regions, and the porous electrically insulating membrane has an electrical conductivity less than the electrical conductivity of the first and second fluidic regions.

Embodiment 4. The cell filtration device of embodiment 3, wherein the pores of the porous electrically insulating membrane are configured to concentrate the inhomogeneous electric field.

Embodiment 5. The cell filtration device of embodiment 4, wherein the pores taper in size from the first membrane surface to the second membrane surface.

Embodiment 6. The cell filtration device of embodiment 4, wherein the pores are uniform or cylindrical in shape.

Embodiment 7. The cell filtration device of embodiment 5, wherein the pores are frustoconical.

Embodiment 8. The cell filtration device of any of embodiments 1 to 7, further comprising a first inlet port configured to introduce the cell suspension into the first fluidic region.

Embodiment 9. The cell filtration device of any of embodiments 1 to 8, wherein the one or more electrodes comprises a first electrode positioned within or adjacent to the first fluidic region, and a second electrode positioned within or adjacent to the second fluidic region.

Embodiment 10. The cell filtration device of embodiment 9, wherein the first electrode is a positive or neutral electrode, and the second electrode is a negative electrode.

Embodiment 11. The cell filtration device of any of embodiments 9 to 10, wherein the first and second electrodes comprise conductive layers on surfaces of rigid substrates.

Embodiment 12. The cell filtration device of any of embodiments 9 to 11, wherein the first and second fluidic regions have a height defined by the first and second electrodes and the porous electrically insulating membrane, and an area defined by contact with the membrane.

Embodiment 13. The cell filtration device of embodiment 12, further comprising a first and second insulating spacer layer connected between and separating each of the first and second electrodes and the porous electrically insulating membrane; wherein the height of the first and second electrodes is defined by a thickness of the first and second insulating layers, and the area of the first and second fluidic regions is defined by a cut-out area of the first and second insulating spacer layers.

Embodiment 14. The cell filtration device of any of embodiments 1 to 10, wherein activation of the one or more electrodes creates a negative dielectrophoretic (DEP) force on the cells.

Embodiment 15. The cell filtration device of embodiment 14, wherein the negative DEP force repels the cells from the porous membrane.

Embodiment 16. The cell filtration device of any of embodiments 1 to 15, wherein the first fluidic region comprises a first channel.

Embodiment 17. The cell filtration device of any of embodiments 1 to 16, wherein the second fluidic region comprises a second channel having a size and shape substantially equivalent to the first channel.

Embodiment 18. The cell filtration device of any of embodiments 1 to 14, wherein the first fluidic region comprises a first fluidic channel and the second fluidic region comprises a second fluidic channel.

Embodiment 19. The cell filtration device of embodiment 18, wherein the first fluidic channel has an inlet and an outlet, and the inlet is fluidically connected to receive a cell suspension from a first vessel, and the outlet of the first fluidic channel is fluidically connected to transport a filtered cell suspension back to the first vessel or to a second vessel.

Embodiment 20. The cell filtration device of embodiment 19, wherein the first fluidic channel comprises at least one additional inlet wherein a second fluid may be introduced to the channel in at least one location.

Embodiment 21. The cell filtration device of embodiment 20, wherein the second fluid comprises a buffer, a wash buffer, or a cell media.

Embodiment 22. The cell filtration device of embodiment 18, wherein the first fluidic region comprises a bioreactor, a tube, or a well in a wellplate.

Embodiment 23. The cell filtration device of embodiment 18, wherein the second fluidic channel is fluidically connected to a waste collection vessel.

Embodiment 24. A method of filtering a cell suspension, the method comprising: providing a cell filtration device comprising: a first fluidic region adapted to receive a cell suspension comprising cells in a medium; a porous electrically insulating membrane having first and second membrane surfaces and a plurality of pores, wherein the first membrane surface of the insulating membrane contacts the first fluidic region; a second fluidic region adapted to receive a liquid through the insulating membrane, wherein the second membrane surface of the insulating membrane contacts the second fluidic region; introducing a cell suspension to the first fluidic region of the cell filtration device, wherein the cell suspension comprises cells in a medium; generating an inhomogeneous electric field across the porous electrically insulating membrane such that the cells in the cell suspension move away from the porous membrane; and passing at least a portion of the medium of the cell suspension through the porous electrically insulating membrane into the second fluidic region.

Embodiment 25. The filtering method of embodiment 24, wherein the inhomogeneous electric field is generated by applying a voltage to one or more electrodes positioned in or adjacent to the first fluidic region and/or the second fluidic region.

Embodiment 26. The filtering method of embodiment 25, wherein the one or more electrodes comprises a first electrode positioned within the first fluidic region, and a second electrode positioned within the second fluidic region.

Embodiment 27. The filtering method of embodiment 25, wherein the voltage is about 25 V or less.

Embodiment 28. The filtering method of embodiment 25, wherein the voltage is from about 1 V to about 5 V.

Embodiment 29. The filtering method of any of embodiments 24 to 28, further comprising creating a pressure differential across the porous electrically insulating membrane in order to drive the medium across the membrane.

Embodiment 30. The filtering method of any of embodiments 24 to 29, further comprising removing the cells from the first fluidic region.

Embodiment 31. The filtering method of any of embodiments 24 to 30, wherein the first fluidic region comprises a first fluidic channel fluidically connected to a first vessel; and the second fluidic region comprises a second fluidic channel fluidically connected to a second vessel.

Embodiment 32. The filtering method of embodiment 31, further comprising continuously passing the cell suspension through the first fluidic channel.

Embodiment 33. The filtering method of embodiment 32, further comprising continuously withdrawing the medium through the second fluidic channel.

Embodiment 34. The filtering method of embodiment 31, wherein the cell filtration device has an inlet and an outlet, and the inlet is fluidically connected to receive a cell suspension from the first vessel, and the outlet of the first fluidic channel is fluidically connected to transport a filtered cell suspension back to the first vessel or to a second vessel.

Embodiment 35. The filtering method of any of embodiments 31 to 34, wherein the first vessel comprises a bioreactor.

Embodiment 36. The filtering method of any of embodiments 24 to 35, wherein the medium passing through the porous electrically insulating membrane further comprises one or more bioproducts, contaminants, wastes, or other components.

Embodiment 37. The filtering method of any of embodiments 24 to 36, wherein the medium comprises a bioproduct, and the method further comprises recovering the bioproduct from the second fluidic region.

Embodiment 38. The filtering method of any of embodiments 24 to 37, further comprising washing the cells, or wherein the cells are concentrated in the first fluidic region.

Embodiment 39. The filtering method of any of embodiments 24 to 38, further comprising adding a fluid to the cell suspension in the first fluidic region.

Embodiment 40. The filtering method of embodiment 39, wherein the added fluid comprises a buffer.

In view of this disclosure it is noted that the methods and apparatus can be implemented in keeping with the present teachings. Further, the various components, materials, structures and parameters are included by way of illustration and example only and not in any limiting sense. In view of this disclosure, the present teachings can be implemented in other applications and components, materials, structures and equipment to implement these applications can be determined, while remaining within the scope of the appended claims.

The above-described embodiments, and particularly any exemplary embodiments, are possible examples of implementations and merely set forth for a clear understanding of the principles of the present invention. Many variations and modifications may be made to the above-described embodiment(s) without substantially departing from the spirit and principles of the techniques described herein. All modifications are intended to be included herein within the scope of this disclosure and protected by the following claims.

1-40. (canceled)

41. A cell filtration device comprising:
a first fluidic region adapted to receive a cell suspension comprising cells in a medium;
a porous electrically insulating membrane having first and second membrane surfaces and a plurality of pores, wherein the first membrane surface of the insulating membrane contacts the first fluidic region;
a second fluidic region adapted to receive a liquid through the pores of the insulating membrane, wherein the second membrane surface of the insulating membrane contacts the second fluidic region; and
one or more electrodes positioned such that application of a voltage to the one or more electrodes will generate an inhomogeneous electric field through the pores of the insulating membrane to repel the cells from the pores and allow the liquid to pass through the pores when the cell suspension is present in the first fluidic region.

42. The cell filtration device of claim 41, wherein the pores of the insulating membrane are configured to concentrate the inhomogeneous electric field, the pores dimensioned to have a tapered shape between the first membrane surface and the second membrane surface or dimensioned to have a uniform or cylindrical shape between the first membrane surface and the second membrane surface, the tapered shape including a frustoconical shape.

43. The cell filtration device of claim 41, wherein the one or more electrodes comprises a first electrode positioned within or adjacent to the first fluidic region, and a second electrode positioned within or adjacent to the second fluidic region.

44. The cell filtration device of claim 43, wherein the first electrode is a positive or neutral electrode, and the second electrode is a negative electrode.

45. The cell filtration device of claim 43, wherein the first and second electrodes comprise conductive layers on surfaces of rigid substrates.

46. The cell filtration device of claim 43, wherein each of the first and second fluidic regions has a height defined by the respective electrode and the respective membrane surface of the insulating membrane.

47. The cell filtration device of claim 46, further comprising a first insulating spacer layer between the first electrode and the first membrane surface, and a second insulating spacer layer between the second electrode and the second membrane surface, wherein the height of each of the first and second fluidic regions is provided by a thickness of the respective insulating spacer layer, and an area of each of the first and second fluidic regions is defined by a cut-out area of the respective insulating spacer layer.

48. The cell filtration device of claim 41, wherein a force that repels the cells from the pores includes a negative dielectrophoretic (DEP) force.

49. The cell filtration device of claim 48, wherein the negative DEP force repels the cells from openings of the pores on the first membrane surface of the insulating membrane.

50. The cell filtration device of claim 41, wherein the first fluidic region comprises a first fluidic channel and the second fluidic region comprises a second fluidic channel.

51. The cell filtration device of claim 50, wherein the first fluidic channel has an inlet and an outlet, and the inlet is fluidically connected to receive a cell suspension from a first vessel, and the outlet of the first fluidic channel is fluidically connected to transport a filtered cell suspension back to the first vessel or to a second vessel.

52. The cell filtration device of claim 51, wherein the first fluidic channel comprises at least one additional inlet configured to allow introduction of a second fluid to the first fluidic channel in at least one location.

53. The cell filtration device of claim 52, wherein the second fluid comprises a buffer, a wash buffer, or a cell media.

54. The cell filtration device of claim 42, wherein the first fluidic region comprises a bioreactor, a tube, or a well in a wellplate.

55. The cell filtration device of claim 50, wherein the second fluidic channel is fluidically connected to a waste collection vessel.

56. A method of filtering a cell suspension, the method comprising:
providing an electrically insulating membrane with a plurality of pores between a first fluidic region and a second fluidic region;
introducing a cell suspension to the first fluidic region, wherein the cell suspension comprises cells in a medium;
generating an inhomogeneous electric field through the pores of the insulating membrane to repel the cells in the cell suspension from the pores; and
passing at least a portion of the medium of the cell suspension through the pores into the second fluidic region.

57. The filtering method of claim 56, wherein the inhomogeneous electric field is generated by applying a voltage to one or more electrodes positioned in or adjacent to the first fluidic region and/or the second fluidic region.

58. The filtering method of claim 57, wherein the one or more electrodes comprises a first electrode positioned within the first fluidic region, and a second electrode positioned within the second fluidic region.

59. The filtering method of claim 58, wherein the voltage includes a voltage difference between the first and second electrodes by a value of 25 V or less.

60. A bioreactor system comprising:
a reactor vessel configured to support a biological process involving a cell suspension having cells in a medium; and
a cell filtration device fluidically connected to the reactor vessel and including a first fluidic region adapted to receive the cell suspension, a porous electrically insulating membrane having first and second membrane surfaces and a plurality of pores, wherein the first membrane surface of the insulating membrane contacts the first fluidic region, the cell filtration device further including a second fluidic region adapted to receive a fluid through the pores of the insulating membrane, wherein the second membrane surface of the insulating membrane contacts the second fluidic region, the cell filtration device further including one or more electrodes positioned such that application of a voltage to the one or more electrodes generates an inhomogeneous electric field through the pores of the insulating membrane to repel the cells from the pores and allow the fluid to pass through the pores when the cell suspension is present in the first fluidic region, wherein the fluid removed through the pores of the insulating membrane includes a desirable bioproduct and/or an undesirable product resulting from the biological process.
